(19) United States
(12) Patent Application Publication
(10) Pub. No.: US 2026/0301910 A1
(43) Pub. Date: Oct. 1, 2026

(54) A COMBINED AND PRACTICAL PRODUCT BASED ON TARGETED EXERCISE, NUTRITION AND MEDICATION

(71) Applicant: Mostafa HADIDIANMOGHADDAM, Zanjan (IR)

(72) Inventor: Mostafa HADIDIANMOGHADDAM, Zanjan (IR)

(21) Appl. No.: 19/477,233

(22) PCT Filed: Oct. 18, 2023
(86) PCT No.: PCT/IB2023/060516

§ 371 Date: Oct. 21, 2025

(51) Int. Cl. G16H 20/30; G16H 20/10; G16H 20/60

(52) U.S. Cl. CPC G16H 20/30; G16H 20/10; G16H 20/60

(57) ABSTRACT

Diabetes, or blood sugar disease, is caused by the resistance of body cells to insulin. The prescribed medicine is prescribed despite the body's resistance to insulin, and this medicine prevents the rise of blood sugar to a large extent, but it does not eliminate insulin resistance, so specialist doctors recommend exercise and proper nutrition to patients when they prescribe the medicine. But this advice is not as precise as medicine and each patient follows it to some extent according to his taste so this advice is not as effective as possible. The imbalance between the three causes diabetes complications, and the more time passes, the more complicated this becomes. In this invention, we control type 1 and 2 diabetes and prevent its complications by strictly implementing the exercise and nutrition program along with the drug therapy administered by the doctor.

The mechanism of diabetes or blood sugar disease due to the resistance of body cells to insulin is activated years before it shows itself in the form of diabetes in the body (high blood sugar in a test or fasting blood sugar test device). It means that many years before a person having diabetes, the body's insulin resistance mechanism begins, and due to not paying attention to the main factor (i.e. the resistance of the body's cells to insulin), the human body begins to secrete insulin automatically through the pancreas. In order to deal with this problem, it is necessary to keep the blood sugar level of the body stable. It means that the body of a person without medication tries to control the normal blood sugar level by secreting extra insulin. And this process is compensated when the person being hungrier and compensating for the hunger caused by insulin secretion eating extra specially sweets and more than the person's actual need. It continues until after a while the body gets tired producing extra insulin secretion then diabetes manifests itself. At first, it shows itself as pre-diabetes and due to lack of attention, it often leads to type 2 diabetes (which, of course, we will find in the following explanations that in the case of practical action and practical training, both type 2 diabetes and type 2 pre-diabetes can be prevented with this educational and practical and combined product with 99.9% certainty.)

In addition, according to the blood sugar status of the diabetic person, the specialist doctor prescribes medicine, either tablets or insulin, or a combination of the two. What the doctor diagnoses and prescribes according to the patient's condition is completely correct and appropriate and there is nothing wrong with it because the disease must be controlled and blood sugar levels should be under control. But what should be noted is that the prescribed medicine is prescribed despite the body's resistance to insulin. This medicine prevents the rise of blood sugar to a large extent but it does not eliminate insulin resistance. Specialist doctors recommend exercise and proper nutrition to patients during drug visits, but this advice is not as precise as medicine, and each patient follows his own taste to some extent. This routine is not as effective and continuous, and this imbalance between the three pillars of medicine, exercise, and nutrition causes the diabetic patient to often rely on medicine, and with the relative observance of other factors, he suffers from diabetes complications in the long term. The more time passes this complexity increases. Why? Because a series of other side effects occur for a diabetic person, which makes these complications more complicated.

### TECHNICAL FIELD

A61K, A61B, A61M

### BACKGROUND ART

AU2019202044B2

System and method for educating users, including responding to patterns

Provided are systems and methods using which users may learn and become familiar with the effects of various aspects of their lifestyle on their health, e.g., users may learn about how food and/or exercise affects their glucose level and other physiological parameters, as well as overall health. In some cases, the user selects a program to try; in other cases, a computing environment embodying the system suggests programs to try, including on the basis of pattern recognition, i.e., by the computing environment determining how a user could improve a detected pattern in some way. In this way, users such as type II diabetics or even users who are only prediabetes or non-diabetic may learn healthy habits to benefit their health.

The difference between this invention and our invention is that a device monitors the drug and other parameters, but in my invention, a person plans and prescribes exercise and nutrition and can change it according to patients' blood sugar level.

U.S. Pat. No. 7,920,998B2

Diabetes care management system

A medical device for diabetes management, comprising (a) a memory comprising one or more optimal blood glucose values, one or more insulin dose values of a patient, one or more measured blood glucose values, and one or more scaling factors for weighting the impact on a future blood glucose value and that are customizable to an individual patient to predict the effect on the blood glucose of insulin dose actions performed by the individual patient, (b) a microprocessor, in communication with the memory, programmed to calculate a further value, the further value being based on the insulin dose values, the optimal blood glucose values, and the scaling factors, and (c) a display configured to display information according to the further value.

In this invention, a device has the task of monitoring the parameters, but in my proposed system, this monitoring is done by a person that is more accurate.

US20210104173A1

Personalized health coaching system

Systems and methods for a comprehensive and personalized approach to health and lifestyle coaching are described. The system may determine health metrics of a user based on detected physiological parameters. The health metrics may be used to determine health recommendations and transmit feedback to the user based on user compliance with the recommendations. This system is coaching but our system is very precise and examined to treat diabetes.

CA2745169C

Diabetes therapy management system

A method of diabetes analysis is provided. A plurality of glucose level readings for a user is received. The plurality of blood glucose level readings is analyzed to generate a report. The report includes a first chart along a 24-hour timeline indicating the plurality of glucose level readings, and a second chart having at least one of infusion device settings and active insulin levels corresponding to the 24-hour timeline of the first chart.

This invention focuses on the blood sugar and blood glucose but our method is combination of diet and exercise to reach the health condition.

US20200268968A1

Methods and systems for managing diabetes

This disclosure relates to a Clinical Decision Support (CDS) system for diabetes management. The CDS system determines a blood glucose level and/or makes a rec-ommendation to an insulin pump parameter based on a plurality of data records rep-resenting one or more predicting factors, e.g., activity data, nutritional information, past blood glucose levels, the rate of change of blood glucose level and/or other contextual data.

### SUMMARY OF INVENTION

Currently, medical treatment for diabetes focuses on controlling blood sugar levels through medication, such as tablets or insulin. While these medications effectively prevent a sharp rise in blood sugar, they do not address the underlying insulin resistance. Specialist doctors also recommend exercise and proper nutrition, but adherence to these lifestyle changes varies among patients. The imbalance between medication, exercise, and nutrition often leads diabetic patients to heavily rely on medication, resulting in long-term complications. Over time, these complications become more complex and are exacerbated by various side effects associated with diabetes. To address this issue, a combined educational and practical product is proposed. This product aims to prevent both type 2 diabetes and pre-diabetes through practical training and action. By providing comprehensive education on the importance of exercise, nutrition, and medication management, this product seeks to empower individuals to effectively manage their blood sugar levels and reduce their reliance on medication.

It is important to note that the proposed product offers a 99.9% certainty in preventing type 2 diabetes and pre-diabetes when implemented correctly. By ad-dressing the root cause of diabetes-insulin resistance- and promoting a balanced approach to treatment, this product aims to improve long-term outcomes for diabetic patients and reduce the complexity of associated complications.

### TECHNICAL PROBLEM

The technical problem in type 2 diabetes is that a diabetic person goes to visit the doctor because of high blood sugar and according to the tests done, the specialist doctor prescribes the right medicine for the patient according to the patient's history and the patient's high sugar status. This decision is completely correct and the blood sugar level of a diabetic person should be reduced and remain low in any way, but this amount of prescribed medicine is due to the body's resistance to insulin not dis-appearing it completely, and this issue is hidden and the prescribed medicine is based on the sugar level. It lowers the blood sugar, but it does not eliminate the body's resistance to insulin, which I have explained in detail before.

The mechanism of type 1 diabetes has differences in some way. The mechanism of type 1 diabetes has differences, because in type 1 diabetes, for some reason, the pancreas gland does not secrete insulin since childhood, and a person with type 1 diabetes must inject insulin to keep his sugar under control at the beginning, but later on, according to the body's physiology, this amount will increase in addition to height and weight changes from childhood to youth. Gradually, the body of a type 1 diabetic person shows resistance to insulin with the same amount of insulin that is actually needed, and the specialist doctor increases the prescription of insulin and other possible drugs according to the increase in the blood sugar of a type 1 diabetic person. Then, the insulin resistance of the body cells should be eliminated with targeted exercise and appropriate and targeted nutrition, and it is definitely better to teach the person from childhood with targeted exercise and targeted nutrition appropriate for that child to implement the medicine prescribed by a specialist doctor is enough to keep the person away from the complications of diabetes from the beginning and its mechanism is similar.

### SOLUTION OF PROBLEM

My invention, which is a combined and practical product centered on targeted exercise for diabetics and targeted nutrition for diabetics in a combined and combined form, is missing in this circle. And that is as a practical program for precise regulation of body cells to eliminate insulin resistance, with the two focus of targeted and specialized exercise for diabetics and targeted nutrition for diabetics, both of which act like airplane wings in control and balance . These two factors plays a vital role in controlling diabetes, and in this balance, the role of medication is very vital, but the amount and type of medication determined after the loss of insulin resistance plays a vital role. All three of these factors cannot eliminate insulin resistance in the body if not followed carefully.

To accurately control diabetes, we need three pillars and supports in a coordinated and adjusted manner:

Medicine column: which should be done individually according to the prescription of the specialist doctor, we are working on two other columns and we will involve this column in the next steps with the coordination of the patient's specialist doctor.

Column of specialized and targeted exercise: every exercise is not suitable for diabetics, including walking, which is not a suitable exercise to affect diabetes and blood sugar, and also in terms of joint damage, especially in people over 45 years old, a diabetic person has to live by walking (for a long time) and his joints are damaged.

### ADVANTAGE EFFECTS OF INVENTION

1—True control of diabetes without body resistance to insulin

2—Diabetes control without bubbles (which is the resistance of body cells)

3—The body adjusts during a process of several months, and the resistance to insulin is continuously eliminated, and the cells of the body respond to insulin, and as a result, with a specific amount of insulin that is in accordance with the real needs of the person, blood sugar can be controlled accurately and without bubbles.

4—The precise adjustment of the body and body cells to eliminate insulin resistance should be precise and calculated and permanent, which means that a person can live with this process permanently.

So far, progress and research have been made in this regard because the results have not been accurate and continuous, and on the other hand, one aspect of research has been given importance, and its effect on the amount of drug control and eliminating the diabetes bubble has been very small, a positive effect for people. It has, but it has not been done so far that the person's medication is reduced and modified and remains permanent.

### BRIEF DESCRIPTION OF DRAWINGS

FIG. 1: Diabetes 1 and 2 flowchart

FIG. 2: Pre diabetes

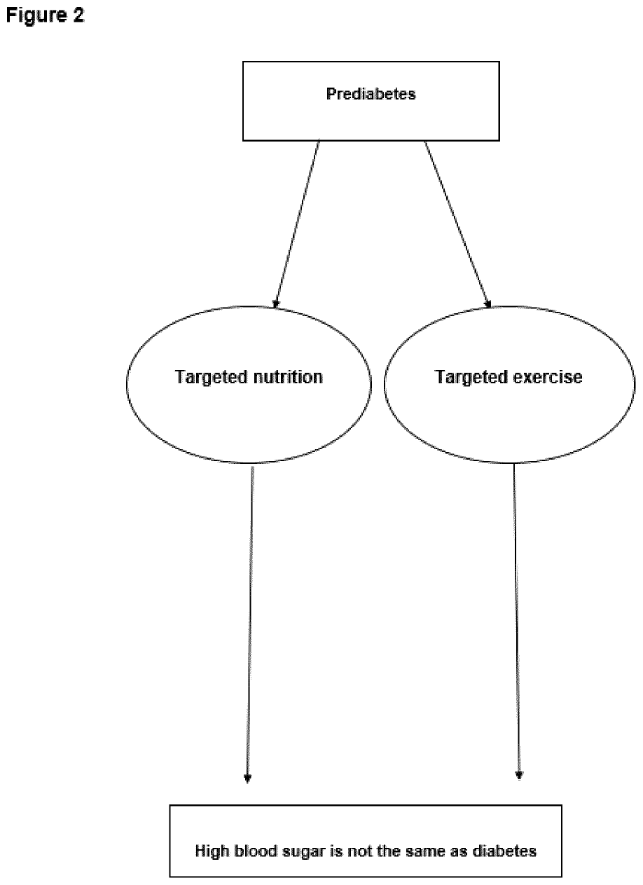

### DESCRIPTION OF EMBODIMENTS

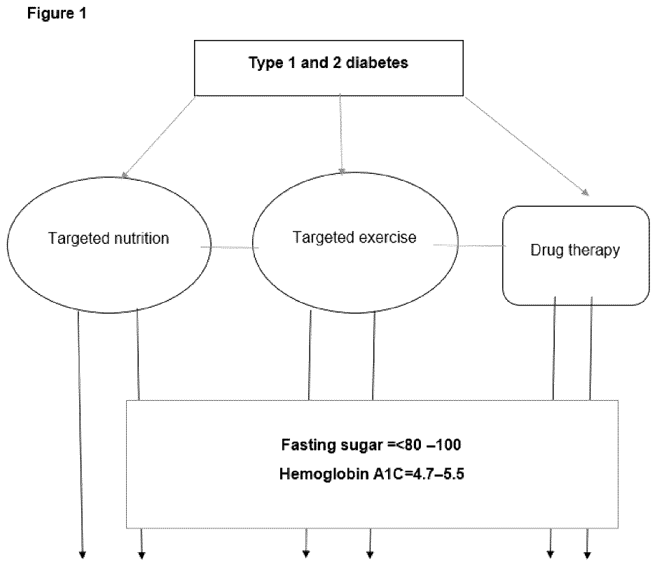

FIG. 1: In figure one there is invented plan for diabetics type 1 and 2. The three segment of diet, exercise and medicine should be under control.

]: In this figure the process for pre diabetic stage is figured. This process is consisting of diet and exercise but no medication. The result of monitoring these two items will be severe drop in blood sugar or hypoglycemia in pre diabetic stage. High blood sugar does not exist as diabetes

### EXAMPLES

1—Normal sugar control for non-diabetic people for 30 months and reduction of medication from insulin to Metformin and Gloripa

2—Weight control from 100 kg to 76 kg

3-Treatment of fatty liver from grade 3 to grade 1 and normalization of liver enzymes

4—Adjusting blood pressure from 13.5 to 11.5 and reducing blood pressure medication from Losartan H to

Losartan 25

5—My kidneys are 30 months old, and they are no longer stone-forming (I removed about 400 kidney stones and had three thiotaxin procedures. Of course, my kidneys did not have any problems in terms of diabetes, and now they are under control, and I no longer have kidney problems.

And I don't have diabetic feet or neuropathy. 6—Other concerns about diabetes heart, diabetes eyes, and kidneys

### INDUSTRIAL APPLICABILITY

Online
Face-to-face and club

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. An industrial product designed to coordinate targeted exercise, targeted nutrition, and physician-supervised targeted medication adjustment, wherein: (a) the targeted exercise and targeted nutrition operate as two synchronized and continuous pillars for metabolic alignment; (b) in individuals with type 1 or type 2 diabetes, medication prescribed by a physician is utilized only after exerting its initial clinical effect, and subsequent adjustment is performed under physician supervision; (c) in non-diabetic or pre-diabetic individuals, metabolic alignment is achieved without medication; and (d) the product provides a measurable, reproducible, and industrially applicable framework for physiological stabilization without delivering any direct medical treatment.

13. The industrial product of claim 12, wherein the targeted exercise pillar comprises structured physical activities that increase muscular glucose utilization and gradually improve insulin sensitivity, said activities being reproducible, scalable, and industrially applicable without imparting any direct therapeutic effect.

14. The industrial product of claim 13, wherein the targeted exercise is configured to create measurable muscular activity preserved over approximately 48-72 hours, and comprising three weekly sessions of 1.5-2.5 hours including aerobic stretching and resistance dumbbell exercises involving major muscle groups.

15. The industrial product of claim 14, wherein the number, type, and intensity of aerobic stretching movements are defined based on measurable individual parameters, and performed independently and safely without conferring any medical treatment.

16. The industrial product of claim 14, wherein the number, type, and intensity of dumbbell-based resistance exercises are defined based on measurable individual parameters, and implemented as a reproducible industrial framework to support metabolic function without therapeutic effect.

17. The industrial product of claim 15, wherein the targeted exercise and targeted nutrition pillars are applied over approximately 4-5 months to achieve measurable metabolic adaptation and stabilization of insulin sensitivity within physiological ranges, independently and without delivering medical treatment.

18. The industrial product of claim 12, wherein the targeted nutrition pillar comprises a structured, data-driven nutritional framework coordinated with the targeted exercise and medication pillars, determining food composition and timing based on energy expenditure, glucose utilization efficiency, and circadian metabolic patterns.

19. The industrial product of claim 16, wherein diabetic users enter a measurable metabolic adaptation phase lasting approximately 4-5 months, during which physiological self-regulation between the targeted exercise pillar and the physician-supervised targeted medication adjustment is established.

20. The industrial product of claim 19, wherein targeted nutrition includes determining food types, quantities, and meal timing to reduce sugar intake, stabilize glucose levels, control consumption of vegetables, seasonal foods, and proteins, and regulate carbohydrate intake in harmony with targeted exercise.

21. The industrial product of claim 20, wherein macronutrient ratios are defined within standard nutritional frameworks, and carbohydrate intake is quantitatively controlled and aligned with the user's energy cycle to maintain a stable metabolic rhythm without therapeutic intervention.

22. The industrial product of claim 12, wherein during supervised use, the prescribed medication is gradually reduced according to the user's insulin resistance profile under physician supervision, while physiological stability remains preserved.

23. The industrial product of claim 12, wherein the product is configured for independent use at home, work, or during travel using portable lightweight components, and requires no clinical or hospital environment, with physician involvement limited solely to medication adjustment.

24. The industrial product of claim 12, wherein the product reduces insulin resistance through targeted exercise and targeted nutrition, and coordinates with physician-supervised targeted medication adjustment in diabetic individuals, while achieving the same physiological regulation in non-diabetic or pre-diabetic individuals without medication.

25. The industrial product of claim 12, wherein diabetic users are eligible for the product only if they are medically supervised by a specialist and have complied with their prescribed medication regimen, and wherein the physician has no involvement in executing the product steps except for supervising medication adjustment.
